(19) United States
(12) Patent Application Publication
(10) Pub. No.: US 2017/0190602 A1
(43) Pub. Date: Jul. 6, 2017

(54) ULTRAVIOLET/OZONE MANIFOLD FOR SWIMMING POOL WATER PURIFICATION

(71) Applicants: Omry Porat, Delray Beach, FL (US); Fernando Figueroa, Boynton Beach, FL (US)

(72) Inventors: Omry Porat, Delray Beach, FL (US); Fernando Figueroa, Boynton Beach, FL (US)

(21) Appl. No.: 15/397,845

(22) Filed: Jan. 4, 2017

Related U.S. Application Data
(60) Provisional application No. 62/387,747, filed on Jan. 4, 2016.

(51) Int. Cl. C02F 1/78; C02F 1/32; C02F 1/00; A61L 2/20

(52) U.S. Cl. CPC C02F 1/78; A61L 2/202; C02F 1/325; C02F 1/008; A61L 2202/11; C02F 2103/42

(57) ABSTRACT

An ozone manifold including a primary flow conduit for directing the pool water through the ultraviolet, ozone manifold. Further, a cross member conduit having an advanced oxidation chamber disposed therein in fluid communication with the primary flow conduit. An inlet flow conduit disposed between the primary flow conduit and the cross member conduit to direct a first bifurcated stream of pool water from the primary flow conduit into a liquid flow chamber within the advanced oxidation chamber. An ozone collection conduit within the advanced oxidation chamber for generating ozone to be mixed with the first bifurcated stream flowing through the inlet flow conduit. An outlet flow conduit disposed between the primary flow conduit and the cross member conduit to direct the first bifurcated stream of pool water from the liquid flow chamber to a water outlet of the flow conduit.

### CROSS-REFERENCE TO RELATED APPLICATIONS

This application claims the benefit of U.S. Provisional Patent Application No. 62/387,747 filed on Jan. 4, 2016, which is incorporated in its entirety herein by reference.

### FIELD OF INVENTION

The present invention relates to swimming pools, and in particular, to an ultraviolet/ozone manifold insertable into a standard conduit system of a swimming pool for the generation of ozone and the introduction of ozone into the pool water, while simultaneously purifying the pool water by means of an ultraviolet light.

### BACKGROUND OF THE INVENTION

### Description of the Prior Art

Recreational swimming pools require a high degree of maintenance. The level of water within the recreational pool must be maintained at a certain level, a skimmer and pumping system must be maintained in order to gather debris which may be floating on the surface of the pool, and the water must be re-circulated through a filter system, and the level of bacteria within the pool must be maintained at a minimum, primarily through the use of chemical additives, such as chlorine . In addition to this, the pool maintenance also includes ‘vacuuming and manual skimming of the surface.

The purification of the water and the maintenance of low levels or reduction of bacteria is one of the most critical considerations by a pool owner, and also one of the most expensive in that the chemicals needed to maintain bacteria free levels are expensive and include chlorine and other similar additives which can be added to the pool in the form of granulated powder, pills, gas injection, or the like.

Various attempts have been made to reduce the levels of chemicals and chlorine needed for water purification and these include the addition of ozone to the water, or in a separate function, the use of ultraviolet light to control bacteria.

### OBJECTS OF THE INVENTION

An object of the present invention to provide for a novel manifold member for insertion within the conduit system of a recreational pool to simultaneously purify re-circulated water utilizing an ultraviolet light, and simultaneously generate ozone for introduction into the fluid stream.

A still further object of the present invention is to provide for a novel manifold member which is easily inserted into the standard conduit system of a recreational pool.

A still further object of the present invention is to generates ozone simultaneously within the conduit system of a recreational pool and to provide for a novel manifold which purifies the water and the swimming pool, and which also contains a monitor to determine if there is sufficient flow through the advanced oxidation chamber, and if not, to automatically extinguish the ultraviolet light source to avoid damage.

A still further object of the present invention is to provide a novel manifold for inclusion in a conduit system of a recreational pool, which will effectively purify the pool water and reduce the costs of chemicals required to maintain water quality.

### SUMMARY OF THE INVENTION

A manifold insertable in the conduit system of a recreational swimming pool for the passage there through of pool water, the pool water flow controlled by a check valve to bifurcate the fluid stream, a first portion of the fluid stream flowing to an ultraviolet light/ozone chamber where the fluid is subjected to ultraviolet light purification, by means of an ultraviolet light positioned within a transparent, watertight sleeve, the ultraviolet light simultaneous forming ozone in the area between the light and sleeve, this ozone being reintroduced into the second bifurcated fluid stream automatically by a low pressure venture affect, this second bifurcated fluid stream being rejoined with the first bifurcated fluid stream for return to the recreational pool.

According to another embodiment of the present invention, there is disclosed an ozone manifold for pool water purification. The ozone manifold includes a primary flow conduit for directing the pool water through the ultraviolet, ozone manifold. Further, a cross member conduit having an advanced oxidation chamber disposed therein in fluid communication with the primary flow conduit. An inlet flow conduit disposed between the primary flow conduit and the cross member conduit to direct a first bifurcated stream of pool water from the primary flow conduit into a liquid flow chamber within the advanced oxidation chamber. An ozone collection conduit within the advanced oxidation chamber for generating ozone to be mixed with the first bifurcated stream flowing through the inlet flow conduit. An outlet flow conduit disposed between the primary flow conduit and the cross member conduit to direct the first bifurcated stream of pool water from the liquid flow chamber to a water outlet of the flow conduit.

According to another embodiment of the present invention, there is disclosed a method of purifying pool water. The method includes directing a first bifurcated stream of pool water from the primary flow conduit to a liquid flow chamber within a Advanced oxidation chamber. Purifying the fluid stream as it flows through a liquid flow chamber with ultraviolet light. Directing a stream of air into an ozone collection conduit within the advanced oxidation chamber. Turning the air stream an ozone collection conduit into ozone with the ultraviolet light. Mixing the ozone from the ozone collection conduit with the first bifurcated stream flowing to the advanced oxidation chamber. Directing the first bifurcated stream from the liquid flow chamber to a water outlet of the primary flow conduit.

### BRIEF DESCRIPTION OF THE DRAWINGS

The structure, operation, and advantages of the present invention will become further apparent upon consideration of the following description taken in conjunction with the accompanying figures (FIGS.). The figures are intended to be illustrative, not limiting. Certain elements in some of the figures may be omitted, or illustrated not-to-scale, for illustrative clarity. The cross-sectional views may be in the form of “slices”, or “near-sighted” cross-sectional views, omitting certain background lines which would otherwise be visible in a “true” cross-sectional view, for illustrative clarity.

In the drawings accompanying the description that follows, both reference numerals and legends (labels, text descriptions) may be used to identify elements. If legends are provided, they are intended merely as an aid to the reader, and should not in any way be interpreted as limiting.

These and other objects of the present invention will become apparent, particularly when taken in light of the following illustrations wherein:

FIG. 1 is a side view ultra/violet, ozone manifold for pool water purification, in accordance with the present invention;

FIG. 2 is a partial side, cutaway view of the advanced oxidation chamber of the ultra/violet, ozone manifold shown in FIG. 1, in accordance with the present invention;

FIG. 3 is an end section view of the advanced oxidation chamber portion of the ultra/violet, ozone manifold taken along Plane 3-3 of FIG. 1, in accordance with the present invention; and

FIG. 4 is a schematic illustration of the operation of the ultra/violet, ozone manifold shown in FIG. 1, in accordance with the present invention.

### DETAILED DESCRIPTION OF THE INVENTION

In the description that follows, numerous details are set forth in order to provide a thorough understanding of the present invention. It will be appreciated by those skilled in the art that variations of these specific details are possible while still achieving the results of the present invention. Well-known processing steps are generally not described in detail in order to avoid unnecessarily obfuscating the description of the present invention.

In the description that follows, exemplary dimensions may be presented for an illustrative embodiment of the invention. The dimensions should not be interpreted as limiting. They are included to provide a sense of proportion. Generally speaking, it is the relationship between various elements, where they are located, their contrasting compositions, and sometimes their relative sizes that is of significance.

In the drawings accompanying the description that follows, often both reference numerals and legends (labels, text descriptions) will be used to identify elements. If legends are provided, they are intended merely as an aid to the reader, and should not in any way be interpreted as limiting.

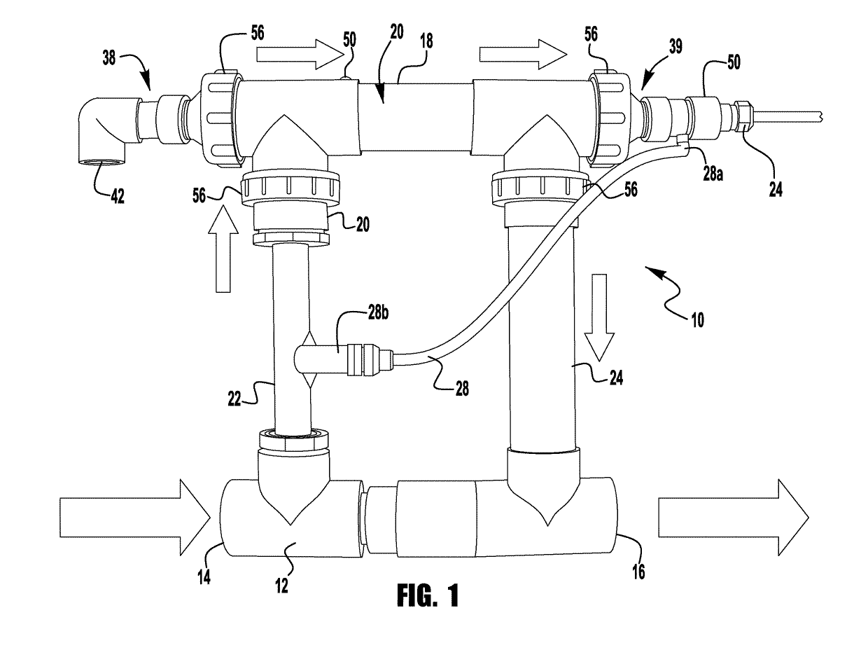

FIG. 1 is a side view of an ultra/violet, ozone manifold 10 for pool water purification. Manifold 10 is designed to be inserted or installed in a standard conduit system utilized with recreational pools with minimal disruption to the conduit system. It should be noted that it is a feature of the present embodiment that all components of the manifold 10 are located within the manifold for easy installation into a pool circuit. Manifold 10 has a primary flow conduit 12 into which untreated water enters and treated water exits. The primary flow conduit 12 has a water inlet 14 into which untreated pool water from a recreational pool conduit system is pumped. The primary flow conduit 12 also includes a treated water outlet 16 which is to be secured to the conduit system of the recreational pool to direct the purified, advanced oxidation enriched pool water back to the pool.

The primary flow conduit 12 is in flow communication with a cross member conduit 18 that includes an advanced oxidation chamber 20. An inlet flow conduit 22 disposed between the primary flow conduit 12 and cross member conduit 18 directs a first bifurcated stream of pool water from water inlet 14 into the cross member conduit 18. An outlet flow conduit 24 is disposed between the cross member conduit 18 and the primary flow conduit 12 to direct treated pool water from the cross member conduit 18 to the primary flow conduit 12 and through the water outlet 16 to the conduit system.

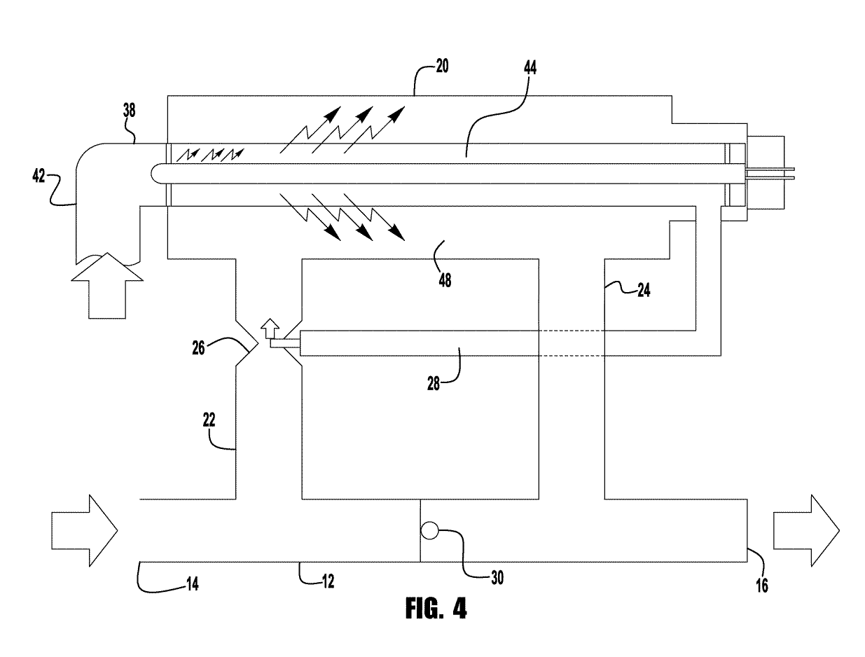

The inlet flow conduit 22 includes a tapered restriction 26, as shown in FIG. 4, that creates a Venturi effect where the pressure downstream of the tapered section decreases and the velocity of the water flowing downstream of the tapered section increases. The increased velocity of the water draws ozone created in the advanced oxidation chamber 20 from an ozone conduit 28 to the section of the interior flow section of the inlet flow conduit 22 downstream from the tapered restriction 26. The ozone is mixed with the first bifurcated stream to provide ozone infused water to the cross member conduit 18.

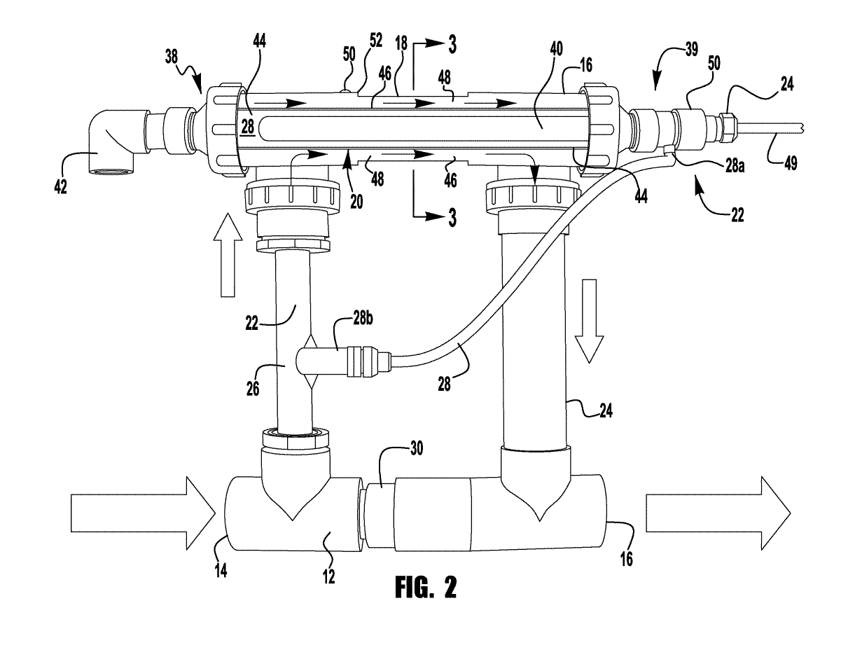

As shown in FIG. 2, an automatic flow regulator 30, such as a check valve, is disposed within the primary flow conduit 12 between the inlet flow conduit 22 and the outlet flow conduit 24. The flow regulator 30 bifurcates the flow of untreated water entering manifold 10 so that a first bifurcated stream of the untreated water flows through flows through the inlet flow conduit 22 and a second bifurcated flow of untreated water flows through the primary conduit towards the water outlet 16.

The first bifurcated stream flowing through inlet flow conduit 22 is infused with ozone and then directed to a liquid flow chamber 48 within the advanced oxidation chamber 20. The liquid flow chamber 48 is provided between the outer wall 52 of the advanced oxidation chamber 20 and a protective transparent sleeve 46 which extends about an ultraviolet bulb 40 extending the length of the advanced oxidation chamber 20. As the ozone infused water flows through the liquid flow chamber 48, it is treated by the Ultraviolet light generated by the ultraviolet bulb 40 to sterilize the water flowing through the liquid flow chamber. After the water is treated in the liquid flow chamber 48, it flows through the outlet flow conduit 24, and out through the water outlet 16 of the primary flow conduit 12.

The second bifurcated stream of untreated water entering the water inlet 14 flows through primary flow conduit 12 and mixes with the treated, first bifurcated stream of water from outlet flow conduit 24 and exits the primary flow conduit 12 through the treated water outlet 16.

As discussed in more detail hereinafter, the use of flow regulator 30 to control the flow volume of the first bifurcated stream of water flowing through the liquid flow chamber 48 in the advanced oxidation chamber 20 is critical to the proper functioning of the ultraviolet sterilization of the pool water. If water moves too quickly through the advanced oxidation chamber 20, the Ultra Violet light generated by an ultraviolet bulb 40, as shown in FIG. 2, does not properly sterilize the water. If water moves too slowly through the liquid flow chamber 48 in advanced oxidation chamber 20, not enough water is sterilized for the treatment of a large body of water like a swimming pool.

The check valve of flow regulator 30 has a preselected tension between 1 lb/sq.in. and 15 lb/sq.in. that causes the check valve to divert a measured flow of the first bifurcated stream of water through the chamber 20 regardless of the horsepower of the pump (not shown) pumping water into the inlet conduit 14 of primary flow conduit 12. This is an important aspect of the invention since it maintains the flow through the UV chamber irrespective of changes in filtration efficiency, which varies according to the amount of debris captured within a water filter (not shown) located upstream of the flow regulator 30.

The advanced oxidation chamber 20 has first and second ends 38 and 39. The first end 38 includes an air inlet 42 connected to a source of air. There is preferably an air filter (not shown) in the air inlet 42. The air inlet 42 is an opening with a range between ¼ inch to 3 inches. The air from air inlet 42 is directed to an ozone collection conduit 44 located between the ultraviolet bulb 40 positioned longitudinally within the advanced oxidation chamber 20 and a protective transparent sleeve 46 which extends about the ultraviolet bulb for the length of the advanced oxidation chamber 20. The light emitted from the ultraviolet bulb 40 interacts with the stream of air from air inlet 42 flowing through the ozone collection conduit 44, to generate and collect a stream of ozone which exits the ozone collection conduit and flows into the ozone conduit 28.

The sleeve 46 shields the ultraviolet bulb 40 from contact with the fluid stream in liquid flow chamber 48 connected to the inlet flow conduit 22 and the outlet flow conduit 24. The protective sleeve 46 is supported by the first and second ends 38 and 39, respectively, of the chamber 20. The protective sleeve 46 is preferably constructed of a glass quartz sleeve that surrounds the bulb 40, and allows for the capture of approximately 100% of the ozone being generated by the UV light from bulb 40 interacting with the air flowing through the ozone collection conduit 44.

The ozone conduit 28 is connected at one end 28a with the ozone collection conduit 44 between the sleeve 46 and the bulb 40 within the advanced oxidation chamber 20 and at the other end 28b with the inlet flow conduit 22 directly downstream from the tapered restriction 26. The restriction 26 forms a conventional venturi which operates to withdraw ozone generated and collected by the bulb 40 within the ozone collection conduit 44 between the sleeve 46 and the bulb. The withdrawn ozone is introduced into the bifurcated fluid stream flowing through inlet flow conduit 22 to the liquid flow chamber 48 in advanced oxidation chamber 20.

The electrical power from a power supply (not shown) is directed through an electrical line 49 into a transformer 50 located at the second end 39 of the advanced oxidation chamber 20 and then to the ultraviolet bulb 40 housed within the advanced oxidation chamber. The perimeter of the power supply is fully illuminated to indicate system status. The color of the perimeter light can be seen from a long distance in order to determine system status. This feature allows the pool owner to view status without manually manipulating menus on a display located on a pool equipment pad usually not easily accessible and tough to see in daylight. Further, the bottom of the power supply has a removable screw allowing access to an isolated weatherproof compartment (IWC). The IWC provides for a conduit connection directly into it isolated from all electronic components.

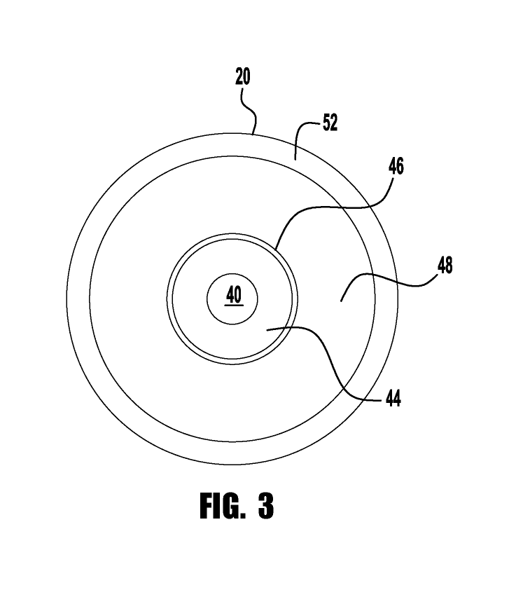

FIG. 3 is a cross section view of the advanced oxidation chamber 20 along Plane 3-3 of FIG. 2 illustrating the ultraviolet bulb 40, the ozone collection conduit 44, the protective sleeve 46, and liquid flow chamber 48.

A temperature sensor 50 is disposed in an outer wall 52 of the manifold 10, as described hereinafter, to avoid stagnation, overheating, and possible fire damage within the chamber 20. The temperature sensor 50 monitors flow through the advanced oxidation chamber 20 and will automatically shut off the ultraviolet bulb 40 when the temperature sensor detects an interruption of water flow, i.e. the temperature in advanced oxidation chamber rises, for any reason. Sensor 50 should be positioned in outer wall 52 of the cross member conduit 18. It should be noted that the sensor 50 may also be a flow sensor or pressure switch.

It should be noted that safety screens, preferably constructed of stainless steel, are disposed at two locations within the manifold 10 to serve as a safeguard against the ultraviolet bulb 40 being broken by debris entering the chamber 20 and glass shards exiting through the outlet conduit 12 into the pool itself. For example, one of the safety screens may be disposed within the water inlet 14 of primary flow conduit 12 and the other safety screen may be disposed within the water outlet 16 of the flow conduit.

In operation, as seen in FIG. 4, the fluid stream from the pool enters into the inlet opening 14 of primary flow conduit 12. The flow is bifurcated with a first bifurcated stream flowing through an inlet flow conduit 22 and the second bifurcated stream flowing through the primary flow conduit 12 and out of outlet opening 16. A check valve 30 disposed in the primary flow conduit 12 controls the volume of the first bifurcated stream flowing through inlet flow conduit 22 and then into a liquid flow chamber 48 within the advanced oxidation chamber 20. The volume of the stream flowing through liquid flow chamber 48 must be regulated to provide for sufficient dwell time within the advanced oxidation chamber 20 to effect purification by the interaction with the ultraviolet light generated by an ultraviolet bulb. Any excess water forming the second bifurcated stream is diverted by the check valve 30 through the outlet opening 16.

Simultaneously, a stream of air enters into an air inlet 42 within a first end 38 of the advanced oxidation chamber 20 and flows through the ozone collection conduit 44. The ultraviolet light interacts with the stream of air flowing through the ozone collection conduit 44 to create a stream of ozone, which exits the ozone collection conduit and flows into the ozone conduit 28. The stream of ozone exits the ozone conduit 28 into inlet flow conduit 22 directly downstream from a tapered restriction 26. The restriction 26 forms a conventional venturi which introduces the withdrawn ozone into the first bifurcated fluid stream flowing through inlet flow conduit 22, whereby ozone infused water enters into the liquid flow chamber 48 in Advanced oxidation chamber 20.

The advanced oxidation chamber 20 operates automatically. If the conduit system of the recreational pool is in operation and fluid is passing through the system from filters to the pool and back to the filters via skimmers or the like, the ultraviolet light bulb 40 will automatically be powered on. If the flow of water to the ultraviolet/ozone chamber 20 is interrupted or diminished such that there is an inadequate flow of water about the protective sleeve 46, a temperature sensor 50 in the outer wall 52 of the ultraviolet ozone chamber will automatically sense this lack of flow or diminished flow because the temperature in the liquid flow chamber 48 will rise and the sensor will extinguish the ultraviolet light 40. This effectively prevents the ultraviolet light bulb 40 from overheating or bursting, because it requires the cooler pool water circulating within the advanced oxidation chamber 20 to provide cooling to prevent the ultraviolet light from overheating.

It will be noted that the various elements of manifold 10 as described heretofore are assembled with a plurality of threaded friction fasteners, all identified by reference numeral 56 which allows maintenance access to the various portions of manifold 10 such as check valve 20 or ultraviolet light bulb 40 for replacement. Therefore, while the present invention has been disclosed with respect to the preferred embodiments thereof, it will be recognized by those of ordinary skill in the art that various changes and modifications can be made without departing from the spirit and scope of the invention. It is therefore manifestly intended that the invention be limited only by the claims and the equivalence thereof.

Although the invention has been shown and described with respect to a certain preferred embodiment or embodiments, certain equivalent alterations and modifications will occur to others skilled in the art upon the reading and understanding of this specification and the annexed drawings. In particular regard to the various functions performed by the above described components (assemblies, devices, etc.) the terms (including a reference to a “means”) used to describe such components are intended to correspond, unless otherwise indicated, to any component which performs the specified function of the described component (i.e., that is functionally equivalent), even though not structurally equivalent to the disclosed structure which performs the function in the herein illustrated exemplary embodiments of the invention. In addition, while a particular feature of the invention may have been disclosed with respect to only one of several embodiments, such feature may be combined with one or more features of the other embodiments as may be desired and advantageous for any given or particular application.

1. An ultraviolet, ozone manifold for pool water purification, comprising;
a primary flow conduit for directing the pool water through the ultraviolet, ozone manifold;
a cross member conduit having an advanced oxidation chamber disposed therein in fluid communication with the primary flow conduit;
an inlet flow conduit disposed between the primary flow conduit and the cross member conduit to direct a first bifurcated stream of pool water from the primary flow conduit into a liquid flow chamber within the advanced oxidation chamber;
an ozone collection conduit within the advanced oxidation chamber for generating and collecting ozone to be mixed with the first bifurcated stream flowing through the inlet flow conduit; and
an outlet flow conduit disposed between the primary flow conduit and the cross member conduit to direct the first bifurcated stream of purified, advanced oxidation enriched pool water from the liquid flow chamber to a water outlet of the flow conduit.

2. The ultraviolet, ozone manifold of claim 1 further including an ultraviolet bulb within the advanced oxidation chamber for purifying the first bifurcated stream of pool water flowing through the liquid flow chamber by the interaction with ultraviolet light.

3. The ultraviolet, ozone manifold of claim 2 wherein the ozone from the ozone collection conduit is mixed with the first bifurcated stream to provide ozone infused water to the liquid flow chamber.

4. The ultraviolet, ozone manifold of claim 3 further including a tapered restriction within the inlet flow conduit to draw ozone created in the ozone collection conduit through an ozone conduit to the inlet flow conduit.

5. The ultraviolet, ozone manifold of claim 1 further including an automatic flow regulator within the primary flow conduit to bifurcate the flow of untreated water entering the manifold so that a first bifurcated stream of water flows through the inlet flow conduit and a second bifurcated flow of water flows through the primary conduit towards the water outlet.

6. The ultraviolet, ozone manifold of claim 5 wherein the automatic flow regulator is a check valve.

7. The ultraviolet, ozone manifold of claim 1 further including a protective transparent sleeve disposed about the ultraviolet bulb extending the length of the advanced oxidation chamber.

8. The ultraviolet, ozone manifold of claim 7 further including the liquid flow chamber provided between an outer wall of the advanced oxidation chamber and the protective transparent sleeve.

9. The ultraviolet, ozone manifold of claim 1 wherein a second bifurcated stream of untreated water entering the water inlet through primary flow conduit mixes with the first bifurcated stream of the purified, advanced oxidation enriched pool water from outlet flow conduit and exits the primary flow conduit through the water outlet.

10. The ultraviolet, ozone manifold of claim 1 further including an air inlet within the advanced oxidation chamber connected to a source of air.

11. The ultraviolet, ozone manifold of claim 10 further including the ozone collection conduit positioned between the ultraviolet bulb positioned longitudinally within the advanced oxidation chamber and the protective transparent sleeve receiving air from the air inlet.

12. The ultraviolet, ozone manifold of claim 11 further including an electrical line directing electrical power to a transformer located at the second end of the advanced oxidation chamber and then to the ultraviolet bulb housed within the advanced oxidation chamber.

13. The ultraviolet, ozone manifold of claim 1 further including a sensor from a group comprising a flow sensor, pressure switch, and temperature sensor disposed in the advanced oxidation chamber to turn off the ultraviolet bulb when the temperature in the advanced oxidation chamber is above a predetermined value.

14. The ultraviolet, ozone manifold of claim 1 further including first and second safety screens disposed at the inlet opening and outlet opening of the primary flow conduit to serve as a safeguard against the ultraviolet bulb being broken by debris entering the chamber and glass shards exiting through the outlet conduit.

15. A method of purifying pool water, including:
directing a first bifurcated stream of pool water from the primary flow conduit to a liquid flow chamber within a advanced oxidation chamber;
purifying the fluid stream as it flows through a liquid flow chamber with ultraviolet light;
directing a stream of air into an ozone collection conduit within the advanced oxidation chamber;
turning the air stream in an ozone collection conduit into ozone with the ultraviolet light;
mixing the ozone from the ozone collection conduit with the first bifurcated stream flowing to the advanced oxidation chamber; and
directing the first bifurcated stream from the liquid flow chamber to a water outlet of the primary flow conduit.

16. The method of claim 15 including drawing ozone created in the ozone collection conduit through an ozone conduit to the inlet flow conduit.

17. The method of claim 16 including bifurcating the flow of untreated water entering the primary flow conduit manifold so that the first bifurcated stream of water flows to the liquid flow chamber and a second bifurcated flow of water flows through the primary conduit towards the water outlet.

18. The method of claim 17 ultraviolet, ozone manifold of claim 16 wherein a second bifurcated stream of untreated water entering the water inlet through primary flow conduit mixes with the first bifurcated stream of water from outlet flow conduit and exits the primary flow conduit through the water outlet.

19. The method of claim 18 including disposing a protective transparent sleeve about the ultraviolet bulb extending the length of the advanced oxidation chamber to form the liquid flow chamber between an outer wall of the advanced oxidation chamber and the protective transparent sleeve.

20. The method of claim 19 including turning off the ultraviolet bulb with a temperature sensor when the temperature in the advanced oxidation chamber reaches a predetermined value.
